(19) United States
(12) Patent Application Publication
(10) Pub. No.: US 2003/0161852 A1
(43) Pub. Date: Aug. 28, 2003

(54) Cosmetic three-phase systems

(75) Inventors: Dennis Miller, Kelkheim (DE); Torsten Henning, Bad Soden (DE)

Correspondence Address: CLARIANT CORPORATION, INTELLECTUAL PROPERTY DEPARTMENT, 4000 MONROE ROAD, CHARLOTTE, NC, 28205, US

(73) Assignee: Clariant GmbH

(21) Appl. No.: 10/338,452

(22) Filed: Jan. 8, 2003

(30) Foreign Application Priority Data
Jan. 11, 2002 (DE) 102 00 724.1

(51) Int. Cl. A61K 7/00

(52) U.S. Cl. 424/401

(57) ABSTRACT

The invention relates to three-phase systems comprising

a) a polyethylene glycol phase comprising

a1) 50-100% by weight of at least one polyethylene glycol and

a2) 0-50% by weight of water,

b) an oil phase and

c) a microemulsion phase comprising

c1) the components of the polyethylene glycol phase a),

c2) the components of the oil phase b) and

c3) at least one surfactant.

The three-phase systems are thermodynamically stable and are preferably suitable as cosmetic compositions, particularly preferably as bath oils.

The phase behavior of hydrocarbon/surfactant/water systems is a constituent of numerous investigations. Through the appropriate choice of components, it is possible to obtain three-phase systems which consist of an upper hydrocarbon phase, a middle microemulsion phase of solubilized hydrocarbon, surfactant and water, and a lower water phase. Such three-phase systems are described in

M. Kahlweit and R. Strey, “The Phase behaviour of H₂O-Oil-Nonionic Amphiphile Ternary Systems” in “Microemulsion Systems”, Surfactant Science Series Vol. 24, Dekker, N.Y., 1987.

K. Shinoda and S. Friberg, “Emulsions and Solubilization”, Wiley, New York, 1986.

H. G. Hauthal and K. Quitsch, “Neues über Mikroemulsionen” [New findings relating to microemulsions], Z. Chem., 30, 274-281 (1990).

In use, such three-phase systems are limited to technical applications and laboratory uses (tertiary petroleum recovery, media for chemical reactions etc.). A reason for this is the toxicity of the upper hydrocarbon phase (toluene etc.), which does not allow their use in the cosmetics and household sector. If the hydrocarbons are replaced by the oils customary in cosmetics (e.g. mineral oils, polydecenes, triglycerides, natural oils and esters), the average microemulsion phase, and thus the entire three-phase system, becomes thermodynamically unstable.

Surprisingly, it has now been found that three-phase mixtures which comprise a polyethylene glycol phase instead of the water phase are thermodynamically stable even when the oil phase consists of the oils customary in cosmetics, such as, for example, mineral oils, polydecenes, triglycerides, natural oils and esters. The polyethylene glycol phase may be a polyethylene glycol/water mixture or pure polyethylene glycols.

The presence of three phases imparts a very esthetic appearance to the three-phase systems according to the invention. By adding dyes, particularly by adding differently colored water-soluble and oil-soluble dyes, it is possible to emphasize the particular external appearance yet further. The use is not limited to the cosmetics and household sector. Use in the industrial sector, in the laboratory sector, but also as a toy, advertising medium, art object, or else as teaching aid for demonstrating physical phenomena is likewise possible.

The invention provides three-phase systems comprising

a) a polyethylene glycol phase comprising

a1) 50-100% by weight of at least one polyethylene glycol and

a2) 0-50% by weight of water,

b) an oil phase and

c) a microemulsion phase comprising

c1) the components of the polyethylene glycol phase a),

c2) the components of the oil phase b) and

c3)at least one surfactant.

Preference is given to three-phase systems which comprise

a) 10 to 80% by weight, preferably 10 to 50% by weight, of the polyethylene glycol phase,

b) 10 to 80% by weight, preferably 10 to 50% by weight, of the oil phase and

c) 10 to 80% by weight, preferably 10 to 50% by weight, of the microemulsion phase.

Preference is given to polyethylene glycol phases a) which comprise 50 to 100% by weight, preferably 75 to 100% by weight, particularly preferably 85 to 100% by weight, of polyethylene glycol and 0 to 50% by weight, preferably 0 to 25% by weight, particularly preferably 0 to 15% by weight, of water.

In a particular embodiment, the polyethylene glycol phase a) comprises 100% by weight of polyethylene glycol.

The polyethylene glycol phase a) can comprise one or more polyethylene glycols.

The polyethylene glycols preferably have a molecular weight of from 150 to 35 000 g/mol, preferably 200 to 800 g/mol.

The surfactants of the microemulsion phase c) may be nonionic, cationic, anionic and/or amphoteric surfactants.

The nonionic surfactants are preferably fatty alcohol ethoxylates, dimethylamine oxides, ethoxylated castor oils, alkyl polyglucosides, fatty acid sorbitol esters, fatty acid polyglycerol esters, ethoxylated fatty acid polyglycerol esters, fatty acid monoethanolamide ethoxylates, glycerol mono- and diesters of fatty acids and/or triesters of phosphoric acid.

Likewise preferred nonionic surfactants are (C₈-C₂₂)-alkyl or alkenyl ethoxylates having 2 to 20 ethylene oxide groups.

The anionic surfactants are preferably monoesters of phosphoric acid, diesters of phosphoric acid, alkyl sulfates, alkyl ether sulfates, preferably sodium laureth sulfate, alkylamidopolyglycol ether sulfates, alkylpolyglycol ether carboxylates, alkylpolyglycol ether sulfosuccinates and/or fatty acid isethionates.

The amphoteric surfactants are preferably acyl glutamates, alkylamidopropylbetaines, preferably cocoamidopropylbetaine, fatty acid methyl taurides, fatty acid sarcosides and/or amphoacetates.

In a particular embodiment, the surfactants are betaines, alkyl ether sulfates or mixtures thereof.

The three-phase systems preferably comprise 1 to 20% by weight, particularly preferably 5 to 20% by weight, of surfactants.

Suitable oil phases are preferably mineral oils, polydecenes, triglycerides, e.g. capric/caprylic triglycerides, natural oils, e.g. orange oil and/or esters, preferably stearates, palmitates and myristates.

Preferred three-phase systems are those in which the components of the oil phase b) are solubilized in the microemulsion phase c) with a degree of solubilization S greater than or equal to 0.8, preferably greater than or equal to 1.5. The degree of solubilization S is the volume ratio of the oil components to the surfactant components.

In a particular embodiment, the three-phase systems also additionally comprise polar organic compounds, preferably hydroxy compounds and/or polyhydroxy compounds, particularly preferably glycerol, propylene glycol, ethanol, hexylene glycol and/or isopropanol.

In a particular embodiment, the three-phase systems also comprise water-soluble and/or oil-soluble dyes which are preferably differently colored. This allows advantageous esthetic effects to be achieved.

The three-phase systems are preferably cosmetic compositions. Particular preference is given to bath oils. Advantageously, the bath oils are shaken prior to use so that they are added to the bath water in the form of an emulsion. Additional ingredients which may be used are the substances customary in cosmetics, such as, for example, perfume oils, ethereal oils, plant extracts, colorants, cationic polymers, solubilization auxiliaries, vitamins and vitamin derivatives, pearlescence-imparting agents, preservatives, skin feel improvers, stabilizers, UV absorbers, hydroxy acids and salts thereof.

Due to the thermodynamic stability of the three-phase systems according to the invention, they can advantageously be prepared by simply mixing the individual components of phases a), b) and c).

The thermodynamic equilibrium of the three phases establishes itself. In a preferred embodiment, the hydrophilic components (polyethylene glycols, water, water-soluble dyes etc.), including the surfactants, are mixed together. Separately from this, the hydrophobic, water-insoluble components (oils, oil-soluble dyes etc.) are mixed together. Subsequently, the hydrophilic mixture and the hydrophobic mixture are mixed together.

Accordingly, the invention also provides three-phase systems obtainable by preparing a mixture comprising

i) polyethylene glycol(s),

ii) optionally water,

iii) oil component(s) and

iv) surfactant(s).

Preference is given to three-phase systems obtainable by preparing a mixture comprising

i) 10 to 70% by weight, preferably 25 to 60% by weight, of polyethylene glycol(s),

ii) 0 to 70% by weight, preferably 0 to 20% by weight, of water,

iii) 10 to 70% by weight, preferably 25 to 60% by weight, of oil component(s) and

iv) 2 to 20% by weight, preferably 5 to 20% by weight, of surfactant(s).

In a particular embodiment, the three-phase systems are free from water. In a preferred embodiment, the mixtures additionally comprise water-soluble and/or oil-soluble dyes.

The preferred polyethylene glycols, oil components and surfactants are the compounds already described above.

### EXAMPLES

In the examples, the degree of ethoxylation of the surfactants was optimized such that approximately equal amounts of oil phase and hydrophilic phase were solubilized in the microemulsion. S is the degree of solubilization of the oil on the basis of the volume ratio. Oleth-8 and Oleth-10 are the INCI names for oleyl alcohol polyglycol ether having 8 or 10 mol, respectively, of ethylene oxide. PEG-8 is the INCI name for polyethylene glycol having an average molar mass of 400 g/mol.

The quantitative data is in % by weight.

### Examples 1 to 3

| | Polyethylene | | | | | | | | |
|---|---|---|---|---|---|---|---|---|---|
| | glycol | | Surfactant | | Oil phase | Phase volume | | | |
| Ex. | PEG-8 | Water | Oleth-8 | Oleth-10 | Mineral oil | Bottom | Middle | Top | S |
| 1 | 43.1 | 10.8 | 6.9 | 0 | 39.2 | 28.9 | 34.8 | 36.3 | 1.5 |
| 2 | 27.2 | 6.8 | 17.3 | 0 | 48.7 | 14.0 | 82.0 | 4.0 | 2.2 |
| 3 | 36.9 | 15.8 | 9.0 | 0.2 | 38.1 | 15.5 | 62.5 | 22.0 | 2.5 |

### Example 4

### Three-Phase Bath Oil

| | Component | % by wt. |
|---|---|---|
| Hydrophilic | PEG-8 | 41.8 |
| components | Water | 10.5 |
| Surfactants | Oleth-5 (® Emulsogen LP) | 4.4 |
| | Coceth-10 ( Genapol C-100) | 5.3 |
| Hydrophobic | Mineral oil | 29.8 |
| components | Capric/caprylic triglycerides | 7.2 |
| | Orange oil | 0.4 |
| | Vitamin E acetate | 0.4 |
| Preservative | Nipaguard PDU | 0.3 |
| Water-soluble dye | Vitasyn Ponceau 4R82 | q.s. |
| Oil-soluble dye | Beta-carotene | q.s |

The bath oil separated into the following three phases:

| | |
|---|---|
| upper phase (red) | 28.5% by volume |
| middle phase (orange) | 48.0% by volume |
| lower phase (yellow) | 23.5% by volume |

The preparation of examples 1 to 4 was carried out by preparing a mixture of the hydrophilic components and, separately, a mixture of the hydrophobic components. Subsequently, the surfactants were added to the hydrophilic mixture. Finally, the hydrophilic mixture and the hydrophobic mixture were mixed together.

1. A cosmetic three-phase system comprising
a) a polyethylene glycol phase comprising
a1) 50-100% by weight of at least one polyethylene glycol and
a2) 0-50% by weight of water,
b) an oil phase and
c) a microemulsion phase comprising
c1) a component of the polyethylene glycol phase a),
c2) a component of the oil phase b) and
c3) at least one surfactant.

2. The cosmetic three-phase system as claimed in claim 1, which comprises
a) 10 to 80% by weight of the polyethylene glycol phase,
b) 10 to 80% by weight of the oil phase and
c) 10 to 80% by weight of the microemulsion phase.

3. The cosmetic three-phase system as claimed in claim 1, wherein the polyethylene glycol phase a) comprises 50 to 100% by weight of polyethylene glycol and 0 to 50% by weight of water.

4. The cosmetic three-phase system as claimed in claim 3, wherein the polyethylene glycol phase a) comprises 100% by weight of polyethylene glycol.

5. The cosmetic three-phase system as claimed in claim 1, wherein the polyethylene glycol has a molecular weight of from 150 to 35 000 g/mol.

6. The cosmetic three-phase system as claimed in claim 1, wherein the surfactant is selected from the group consisting of nonionic surfactants, cationic surfactants, anionic surfactants, amphoteric surfactants, and mixtures thereof.

7. The cosmetic three-phase system as claimed in claim 6, wherein the nonionic surfactants are selected from the group consisting of fatty alcohol ethoxylates, dimethylamine oxides, ethoxylated castor oils, alkyl polyglucosides, fatty acid sorbitol esters, fatty acid polyglycerol esters, ethoxylated fatty acid polyglycerol esters, fatty acid monoethanolamide ethoxylates, glycerol mono- and diesters of fatty acids, triesters of phosphoric acid, and mixtures thereof.

8. The cosmetic three-phase system as claimed in claim 6, wherein the nonionic surfactants are (C₈-C₂₂)-alkyl or alkenyl ethoxylates having 2 to 20 ethylene oxide groups.

9. The cosmetic three-phase system as claimed in claim 6, wherein the anionic surfactants are selected from the group consisting of monoesters of phosphoric acid, diesters of phosphoric acid, alkyl sulfates, alkyl ether sulfates, preferably sodium laureth sulfate, alkylamidopolyglycol ether sulfates, alkylpolyglycol ether carboxylates, alkylpolyglycol ether sulfosuccinates, fatty acid isethionates, and mixtures thereof.

10. The cosmetic three-phase system as claimed in claim 6, wherein the amphoteric surfactants are selected from the group consisting of acyl glutamates, alkylamidopropylbetaines, fatty acid methyl taurides, fatty acid sarcosides, amphoacetates, and mixtures thereof.

11. The cosmetic three-phase system as claimed in claim 6, wherein the surfactants are selected from the group consisting of betaines, alkyl ether sulfates, and mixtures thereof.

12. The cosmetic three-phase system as claimed in claim 1, wherein the component of the oil phase b) is selected from the group consisting of mineral oils, polydecenes, triglycerides, natural oils, esters, and mixtures thereof.

13. The cosmetic three-phase system as claimed in claim 12, wherein the component of the oil phase b) is solubilized in the microemulsion phase c) with a degree of solubilization S greater than or equal to 0.8.

14. The cosmetic three-phase system as claimed in claim 1, which further comprises polar organic compounds selected from the group consisting of hydroxy compounds, polyhydroxy compounds, and mixtures thereof.

15. The cosmetic three-phase system as claimed in claim 1, which further comprises at least one water-soluble dye.

16. The cosmetic three-phase system as claimed in claim 1, which further comprises at least one oil-soluble dye.

17. The cosmetic three-phase system as claimed in claim 1, which further comprises at least one water-soluble dye and at least one oil-soluble dye.

18. The cosmetic three-phase system as claimed in claim 17, wherein the water-soluble dyes and the oil-soluble dyes are differently colored.

19. The cosmetic three-phase system as claimed in claim 1 in the form of a cosmetic composition.

20. The cosmetic three-phase system as claimed in claim 19, in the form of a bath oil.

21. A cosmetic three-phase system obtained by preparing a mixture comprising
i) polyethylene glycols;
ii) optionally water;
iii) oil components; and
iv) surfactants.

22. A cosmetic three-phase system as claimed in claim 21, obtained by preparing a mixture comprising
i) 10 to 70% by weight of polyethylene glycol,
ii) 0 to 70% by weight of water,
iii) 10 to 70% by weight of oil components and
iv) 2 to 20% by weight of surfactants.

23. The cosmetic three-phase system as claimed in claim 22, wherein the polyethylene glycols i) have a molecular weight of from 150 to 35 000 g/mol.

24. The cosmetic three-phase system as claimed in claim 22, wherein the oil components iii) are selected from the group consisting of mineral oils, polydecenes, triglycerides, natural oils, esters, and mixtures thereof.

25. The cosmetic three-phase system as claimed in claim 22, which additionally comprises water-soluble and/or oil-soluble dyes being differently colored.

26. The cosmetic three-phase system as claimed in claim 22, in the form of a cosmetic composition.

27. The cosmetic three-phase system as claimed in claim 1, which comprises
a) 10 to 50% by weight, of the polyethylene glycol phase,
b) 10 to 50% by weight, of the oil phase and
c) 10 to 50% by weight, of the microemulsion phase.

28. The cosmetic three-phase system as claimed in claim 1, wherein the polyethylene glycol phase a) comprises 75 to 100% by weight of polyethylene glycol and 0 to 25% by weight of water.

29. The cosmetic three-phase system as claimed in claim 1, wherein the polyethylene glycol phase a) comprises 85 to 100% by weight of polyethylene glycol and 0 to 15% by weight of water.

30. The cosmetic three-phase system as claimed in claim 1, wherein the polyethylene glycol has a molecular weight of 200 to 800 g/mol.

31. The cosmetic three-phase system as claimed in claim 1, wherein the amphoteric surfactants comprise cocoamidopropylbetaine.

32. The cosmetic three-phase system as claimed in claim 1, wherein the component of the oil phase b) is a capric triglyceride and/or a caprylic triglyceride.

33. The cosmetic three-phase system as claimed in claim 1, wherein the component of the oil phase b) is an orange oil.

34. The cosmetic three-phase system as claimed in claim 1, wherein the component of the oil phase b) is an ester selected from the group consisting of stearates, palmitates, mystistates, and mixtures thereof.

35. The cosmetic three-phase system of claim 12 wherein the degree of stabilization S is greater than or equal to 1.5.

36. The cosmetic three-phase system of claim 14 wherein the polar organic compounds are selected from the group consisting of glycerol, propylene glycol, ethanol, hexylene glycol, isopropanol, and mixtures thereof.

37. The cosmetic three-phase system of claim 22 which is obtained by preparing a mixture comprising:
i) 25-60 weight percent polyethylene glycol;
ii) 0-20 weight percent water;
iii) 25-60 weight percent oil components; and
iv) 5-20 weight percent surfactants.

38. The cosmetic three-phase system of claim 22 wherein the polyethylene glycols i) have a molecular weight of from 200 to 800 g/mol.

39. The cosmetic three-phase system of claim 22 wherein the oil components ii) comprise capric and/or capryilic tryglycerides.

40. The cosmetic three-phase system of claim 22 wherein the oil components ii) comprise orange oil.

41. The cosmetic three-phase system of claim 22 wherein the oil components ii) are selected from the group consisting of stearates, palmitates, myristates, and mixtures thereof.

42. A bath oil comprising the cosmetic three-phase system of claim 1.

43. The cosmetic three-phase system of claim 1, wherein said cosmetic three-phase system is free of a water phase.

44. The cosmetic three-phase system of claim 21, wherein said cosmetic three-phase system is free of a water phase.

45. A method for preparing bath water comprising shaking the cosmetic three-phase system of claim 1 to form an emulsion and adding the emulsion to the bath water.
